(19) United States
(12) Patent Application Publication
(10) Pub. No.: US 2026/0069154 A1
(43) Pub. Date: Mar. 12, 2026

(54) CUFF AND BLOOD PRESSURE MEASUREMENT DEVICE HAVING THE SAME

(71) Applicant: CHARMCARE CO., LTD., Seoul (KR)

(72) Inventor: Dong Hwa LEE, Yongin-si (KR)

(21) Appl. No.: 19/393,179

(22) Filed: Nov. 18, 2025

Related U.S. Application Data
(63) Continuation of application No. PCT/KR2023/018021, filed on Nov. 10, 2023.

(30) Foreign Application Priority Data
May 19, 2023 (KR) 10-2023-0064862

(51) Int. Cl. A61B 5/022; A61B 5/00

(52) U.S. Cl. CPC A61B 5/02233; A61B 5/6824; A61B 5/7445

(57) ABSTRACT

A cuff for a blood pressure measuring device includes: an air supply unit comprising an air inlet through which air is introduced from the outside and an air outlet through which air inside is discharged; a pressurizing unit connected to the air supply unit and configured to expand when air is introduced from the air supply unit, and a folding portion disposed between the air supply unit and the pressurizing unit and including a first air passage that connects the air supply unit and the pressurizing unit to communicate with each other. The pressurizing unit includes a main air bladder disposed to contact the wrist and connected to the air supply unit, at least one sub air bladder laminated on one side of the main air bladder, and a communication passage that connects the main air bladder and the sub air bladder to communicate with each other.

### BACKGROUND

### Field

The present invention relates to a cuff for a blood pressure measuring device and a blood pressure measuring device including the same.

Blood pressure information is one of the important indicators for assessing a person’s health condition. Blood pressure refers to the pressure exerted by blood on the walls of blood vessels. When the heart contracts to pump blood, the pressure in the blood vessels is referred to as systolic pressure or maximum blood pressure, and when the heart relaxes to receive blood, the pressure in the blood vessels is referred to as diastolic pressure or minimum blood pressure.

Generally, in a healthy person, the systolic pressure is below 120 mmHg and the diastolic pressure is below 80 mmHg. Meanwhile, a person whose systolic pressure is 140 mmHg or higher and diastolic pressure is 90 mmHg or higher is considered to have hypertension, and a person whose systolic pressure is 90 mmHg or lower and diastolic pressure is 60 mmHg or lower is considered to have hypotension.

Since abnormal blood pressure conditions, such as hypertension or hypotension, may indicate health problems, it is important to measure blood pressure in order to obtain accurate blood pressure information. Typical methods for measuring blood pressure include auscultatory, oscillometric, and tonometric methods.

Among these, the oscillometric method measures systolic and diastolic pressure by detecting pulse waves that occur when the blood flow in an artery is temporarily blocked by pressurizing a part of the body through which arterial blood flows, followed by gradual depressurization at a constant rate, or by detecting pulse waves generated during pressurization at a constant rate.

The tonometric method, on the other hand, measures blood pressure by applying a constant pressure to a body part such that the blood flow in the artery is not completely blocked, and determining the blood pressure using the amplitude and waveform of the pulse generated at that time.

Conventional blood pressure measuring devices employing the oscillometric method are generally configured to be wound around a subject’s arm. However, such arm-type blood pressure measuring devices are bulky and inconvenient to carry.

To address this issue, Korean Registered Patent No. 10-1349767 (hereinafter referred to as “the prior document”) discloses a wristwatch-type blood pressure monitor configured to be worn on a subject’s wrist, thereby improving portability, and including an air chamber capable of pressurizing the subject’s artery. In the prior document, when the air chamber presses the subject’s artery, the pressure of the air chamber can be measured, and the subject’s blood pressure can be calculated based on the measured air chamber pressure.

However, the larger the amount of air accommodated in the air chamber, the more accurately the pressure of the air chamber can be measured. If an insufficient amount of air is accommodated, measurement errors may occur in determining the pressure of the air chamber.

Although the prior document discloses an air chamber capable of accommodating air, it does not describe a specific structure of the air chamber that allows for accommodating the maximum possible amount of air.

Moreover, the larger the contact area between the expanded air chamber and the wrist, the more effectively the artery of the subject can be pressurized. However, in the prior document, the air chamber expands in a balloon-like shape when air is injected therein, which limits the effective increase of the contact area between the air chamber and the wrist.

### SUMMARY

The present invention aims to solve the problem in which errors occur in blood pressure measurement due to a low pressure in the air bladder of a cuff.

Another object of the present invention is to solve the problem in which the flow passage within the foldable portion of the cuff is bent, thereby hindering smooth movement of air.

It should be noted, however, that the technical problems to be achieved by the present embodiment are not limited to the aforementioned examples, and other technical problems may also be present.

As a technical means for solving the above-described technical problems, a cuff for a blood pressure measuring device according to a first embodiment of the present invention includes: an air supply unit including an air inlet through which air is introduced from the outside and an air outlet through which air inside is discharged; a pressurizing unit connected to the air supply unit, which expands when air is introduced from the air supply unit to pressurize a radial artery located at the wrist of a subject; and a folding portion disposed between the air supply unit and the pressurizing unit, and including a first air passage that connects the air supply unit and the pressurizing unit to communicate with each other. The pressurizing unit is disposed to contact the wrist of the subject and includes: a main air bladder connected to the air supply unit; at least one sub air bladder laminated on one side of the main air bladder; and a communication passage that connects the main air bladder and the sub air bladder to communicate with each other. In this case, a cross-sectional area of the main air bladder is formed larger than that of the sub air bladder.

The cuff may further include a stent located inside the first air passage of the folding portion, the stent being formed in a coil spring shape.

The cuff may further include: a first branch passage that connects the air supply unit and the first air passage, the first branch passage branching into two flow paths from one end of the first air passage to connect to the air supply unit; and a second branch passage that connects the first air passage and the main air bladder, the second branch passage branching into two flow paths from the other end of the first air passage to connect to the main air bladder.

The cuff may further include at least one auxiliary air bladder communicating with the air supply unit and located in a direction opposite to the first air passage.

The folding portion may have a margin in length so as to be bent into an S-shape when coupled to a strap for wrist wearing.

A blood pressure measuring device according to a second embodiment of the present invention includes: a first strap configured to wrap around a portion of a subject’s wrist adjacent to an ulnar artery; a second strap connected to the first strap and configured to wrap around a portion of the wrist adjacent to a radial artery, the second strap including a cuff; and a main body installed on the first or second strap. The main body includes: a pump unit that supplies air to the cuff or suctions air from the cuff to discharge it; a measurement unit that measures the pressure of the cuff; and a control unit that calculates the blood pressure of the user based on the pressure measured by the measurement unit.

The main body may further include a display unit configured to display the calculated blood pressure.

The main body may further include a communication unit configured to transmit and receive data to and from an external device.

According to the means for solving the problems described above, the cuff for a blood pressure measuring device of the present invention forms the cross-sectional area of the main air bladder, which is adjacent to the subject’s wrist, larger than that of the sub air bladder, so that the main air bladder is preferentially expanded. This allows the radial artery located at the wrist of the subject to be pressurized with sufficient pressure, thereby enabling accurate measurement of blood pressure.

In addition, since a stent is disposed inside the air passage of the folding portion of the cuff, even when the folding portion is folded, the passage is prevented from being blocked, thereby ensuring reliable air flow.

### BRIEF DESCRIPTION OF THE DRAWINGS

FIG. 1 is a perspective view of a cuff for a blood pressure measuring device according to an embodiment of the present invention.

FIG. 2 is a cross-sectional view taken along line A–A′ of FIG. 1.

FIG. 3 is a diagram illustrating a pressurizing unit according to an embodiment of the present invention.

FIG. 4A is a plan view of the pressurizing unit according to an embodiment of the present invention, and FIG. 4B is a bottom view of the pressurizing unit according to an embodiment of the present invention.

FIG. 5 is a schematic diagram of a blood pressure measuring device according to an embodiment of the present invention.

FIG. 6 is a block diagram illustrating a main body of the blood pressure measuring device according to an embodiment of the present invention.

### Description of reference characters

10 : Blood pressure measuring device

100 : Cuff

110 : Air supply unit

111 : Air inlet 112 : Air outlet

120 : Pressurizing unit

121 : Main air bladder

122 : Sub air bladder

123 : Communication passage

130 : Folding portion

131 : First air passage

132 : First branch passage

133 : Second branch passage

140 : Stent

150 : Auxiliary air bladder

200 : First strap

300 : Second strap

310 : First Velcro fastener 320 : Second Velcro fastener

400 : Main body

410 : Pump unit

420 : Measurement unit

430 : Control unit

440 : Display unit

460 : Power supply unit

470 : Communication unit

### DETAILED DESCRIPTION

Hereinafter, exemplary embodiments of the present invention will be described in detail with reference to the accompanying drawings so that those skilled in the art to which the present invention pertains can easily carry out the invention. However, the present invention may be implemented in various different forms and is not limited to the embodiments described herein. For clarity, parts not related to the description are omitted in the drawings, and like reference numerals are used to designate like elements throughout the specification.

In the entire specification of the present application, when it is stated that one part is “connected” to another part, this includes not only cases where the parts are “directly connected” to each other, but also cases where the parts are “electrically connected” to each other via another element interposed therebetween.

In the entire specification of the present application, when it is stated that a certain member is “on” another member, this includes not only cases where the member is in direct contact with the other member, but also cases where another member is interposed between them.

Hereinafter, embodiments of the present invention will be described in detail with reference to the accompanying drawings.

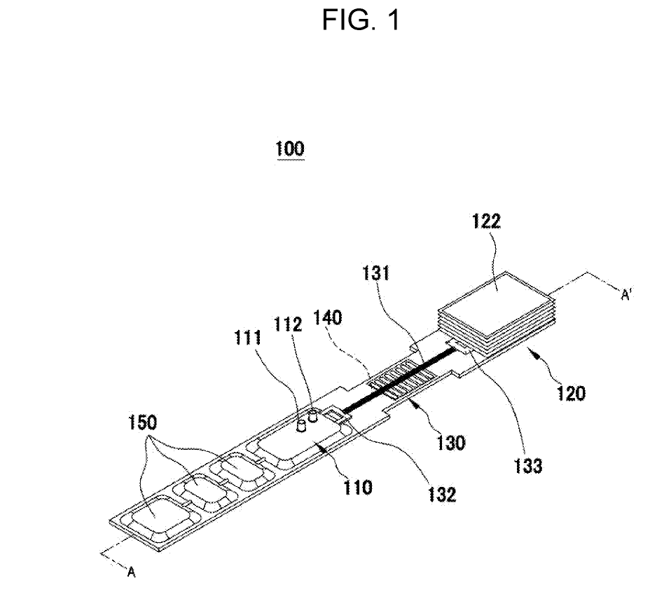

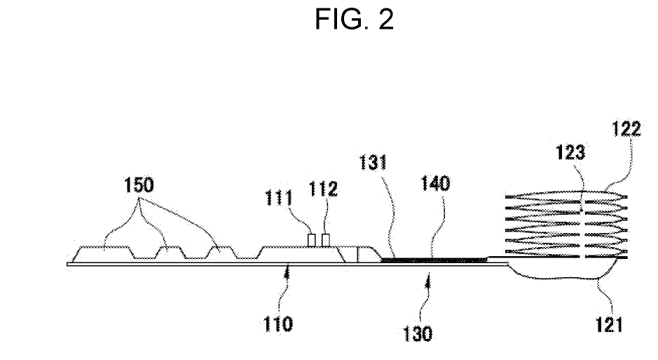

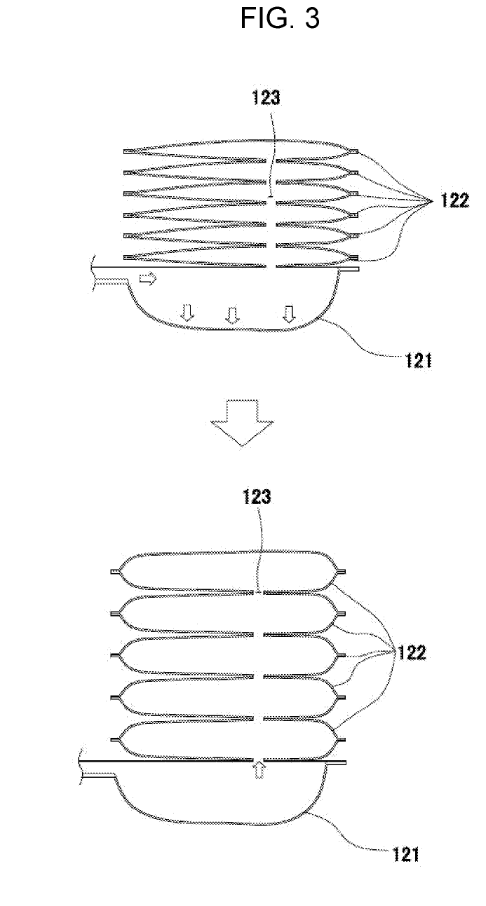

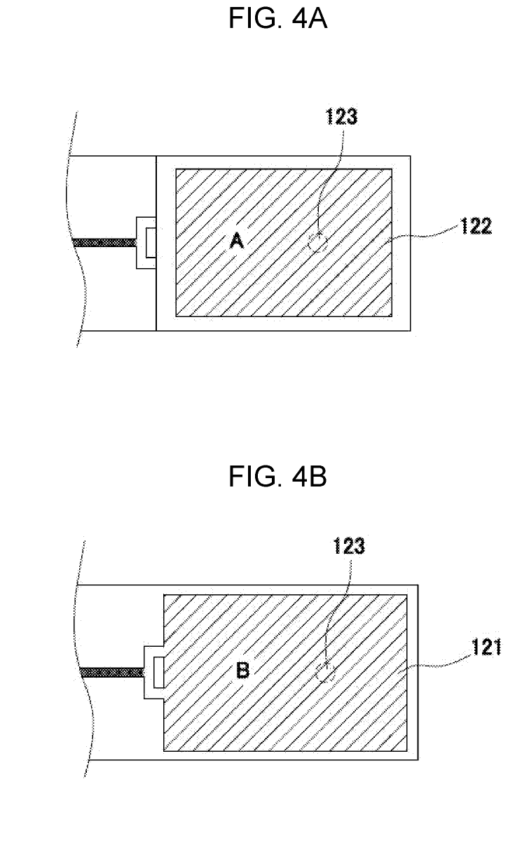

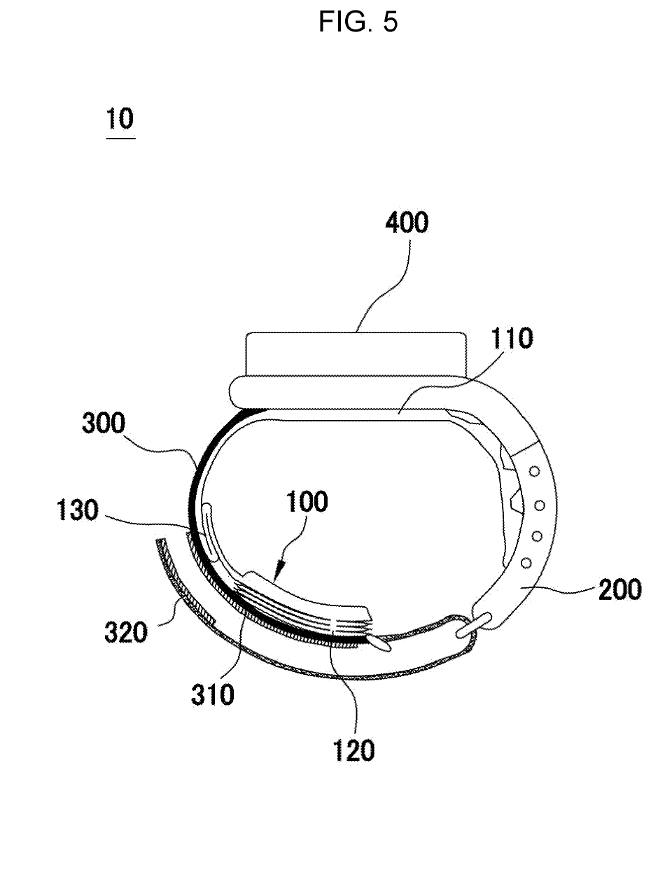

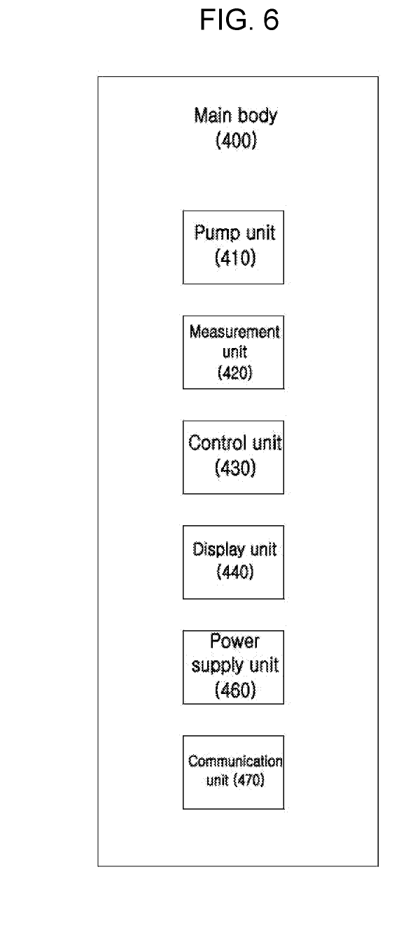

FIG. 1 is a perspective view of a cuff for a blood pressure measuring device according to an embodiment of the present invention, FIG. 2 is a cross-sectional view taken along line A–A′ of FIG. 1, FIG. 3 is a diagram illustrating a pressurizing unit according to an embodiment of the present invention, FIG. 4A is a plan view of the pressurizing unit according to an embodiment of the present invention, FIG. 4B is a bottom view of the pressurizing unit according to an embodiment of the present invention, FIG. 5 is a schematic diagram of a blood pressure measuring device according to an embodiment of the present invention, and FIG. 6 is a block diagram illustrating a main body of the blood pressure measuring device according to an embodiment of the present invention.

Hereinafter, with reference to FIGS. 1 to 4B, a cuff 100 of a blood pressure measuring device 10 according to an embodiment of the present invention hereinafter referred to simply as “cuff 100” will be described.

The cuff 100 includes an air supply unit 110, a pressurizing unit 120, and a folding portion 130.

The air supply unit 110 includes an air inlet 111 through which air is introduced from the outside and an air outlet 112 through which air inside is discharged. In other words, the air supply unit 110 is connected to a main body 400 of the blood pressure measuring device 10 described later, and functions to supply air from a pump unit 410 of the main body 400 to the cuff 100 or discharge air from the cuff 100. For example, the air supply unit 110 has a predetermined space capable of accommodating introduced air, and the introduced air may be transferred to the pressurizing unit 120 through a first air passage 131 of the folding portion 130.

The pressurizing unit 120 is connected to the air supply unit 110 and expands when air is introduced from the air supply unit 110, thereby pressurizing the radial artery located at the wrist of a subject. The pressurizing unit 120 is formed by stacking a plurality of air bladders 121, 122 in a bellows shape, and each of the air bladders 121, 122 may communicate with each other. In addition, the pressurizing unit 120 may be made of an elastic material such as silicone, urethane, or rubber that expands when air is injected inside and contracts when the injected air is discharged. A detailed description of the pressurizing unit 120 will be given below.

The folding portion 130 is disposed between the air supply unit 110 and the pressurizing unit 120, and includes a first air passage 131 that connects the air supply unit 110 and the pressurizing unit 120 to communicate with each other.

When the cuff 100 is mounted on the blood pressure measuring device 10 for use, the folding portion 130 allows the pressurizing unit 120 to contact a region of the user’s wrist where the radial artery is located. To this end, the folding portion 130 may have an extra length and may be bent into an S-shape so that the overall length of the folding portion 130 can be adjusted, thereby enabling adjustment of the position of the pressurizing unit 120. The bent configuration of the folding portion 130 can be seen in FIG. 4.

The folding portion 130 may further include a stent 140 located inside the first air passage 131 and formed in a coil spring shape. Since the folding portion 130 is installed in a bent S-shape, the stent 140, which is cylindrical, may be positioned inside the first air passage 131 to prevent the air passage from being blocked at the bent part, thereby forming a flow path through which air can pass inside the stent 140. For example, the stent 140 may be made of a metal material such as stainless steel, but is not limited thereto.

Referring to FIG. 1, the cuff 100 may further include a first branch passage 132 and a second branch passage 133. The first branch passage 132 connects the air supply unit 110 and the first air passage 131, branching into two flow paths from one end of the first air passage 131 to connect to the air supply unit 110. The second branch passage 133 connects the first air passage 131 and the main air bladder 121, branching into two flow paths from the other end of the first air passage 131 to connect to the main air bladder 121. In other words, the branch passages 132, 133 are respectively positioned at both ends of the first air passage 131, allowing air supplied from the air supply unit 110 to flow smoothly into the first air passage 131 and to be efficiently supplied to the main air bladder 121. When discharging air from the pressurizing unit 120, the air can move through the first and second branch passages 132, 133 to the air supply unit 110 for discharge.

Hereinafter, the pressurizing unit 120 according to an embodiment of the present invention will be described with reference to FIGS. 3 to 4B.

Referring to FIG. 3, the pressurizing unit 120 includes a main air bladder 121, at least one sub air bladder 122, and a communication passage 123.

The main air bladder 121 is positioned at a portion where the subject’s wrist is located and is connected to the air supply unit 110 to receive air therefrom. For example, the portion of the main air bladder 121 that contacts the subject’s wrist may be formed to protrude, and when air is introduced, the main air bladder 121 expands outwardly from the inside.

A cross-sectional area of the main air bladder 121 may be formed larger than that of the sub air bladder 122. The term “cross-sectional area” refers to the area, viewed from above, of the portion of the air bladder 121, 122 that expands when air is introduced into the air bladder before inflation. For example, as shown in FIG. 4, the cross-sectional area B of the main air bladder 121 may be larger than the cross-sectional area A of the sub air bladder 122.

Accordingly, as shown in FIG. 3, the air introduced into the main air bladder 121 does not immediately flow into the sub air bladder 122, but rather, after the main air bladder 121 expands, moves through the communication passage 123 and is supplied to the sub air bladder 122. In this way, the pressurizing unit 120 allows air to be injected into the sub air bladder 122 only after the main air bladder 121 has first expanded. In the process of measuring blood pressure according to the present invention, the pressure of the air introduced into the air bladder increases sequentially. At an early stage when the air pressure is low, the pressurizing unit 120 may not sufficiently pressurize the radial artery. Furthermore, due to the weight or resistance of multiple sub air bladders 122, the main air bladder 121 may have difficulty expanding easily. To address this, the main air bladder 121 is configured to expand preferentially, thereby enabling the pressurizing unit 120 to effectively pressurize the radial artery of the subject’s wrist even when the air pressure is low.

A plurality of sub air bladders 122 are laminated on one side of the main air bladder 121. The sub air bladders 122 may have smaller cross-sectional areas than the main air bladder 121, as described above. In other words, when air is injected into and inflates the main air bladder 121 and the sub air bladders 122, each sub air bladder 122 is formed such that its expanded volume is smaller than that of the main air bladder 121.

The communication passage 123 connects the main air bladder 121 and the plurality of sub air bladders 122 so that they communicate with each other. For example, the communication passage 123 may be formed by perforating a hole in the upper surface of the main air bladder 121, and by forming holes in the upper and lower surfaces of each sub air bladder 122 at positions corresponding to the hole of the main air bladder 121. In this case, the sub air bladder 122 positioned at the topmost layer may have a hole perforated only on its lower surface.

Referring again to FIG. 1, the cuff 100 may further include at least one auxiliary air bladder 150 that communicates with the air supply unit 110 and is located in a direction opposite to the first air passage 131. When the cuff 100 is mounted on the blood pressure measuring device 10, the auxiliary air bladder 150 may be positioned on the inner side of a first strap 200, so that the user’s wrist does not directly contact the relatively rigid material of the first strap 200. Instead, the wrist comes into contact with the air-filled auxiliary air bladder 150, thereby minimizing the load on the wrist during blood pressure measurement.

Hereinafter, with reference to FIGS. 4 and 5, a blood pressure measuring device 10 according to an embodiment of the present invention will be described.

The blood pressure measuring device 10 may be configured to be wearable on a subject’s wrist, and includes a first strap 200 that wraps around a portion of the wrist of the subject, a second strap 300 that wraps around another portion of the wrist, and a main body 400 that calculates the blood pressure.

The first strap 200 is disposed to wrap around a portion of the subject’s wrist adjacent to the ulnar artery and is formed of a material having a predetermined level of rigidity or greater, thereby preventing the wrist and the ulnar artery from being pressed by the first strap 200 and ensuring that the blood flow through the ulnar artery is not obstructed.

The second strap 300 is connected to the first strap 200 to wrap around another portion of the wrist adjacent to the radial artery, and includes the cuff 100. For example, the second strap 300 may have a first Velcro fastener 310 attached at a middle portion and a second Velcro fastener 320 attached at an end portion, allowing the blood pressure measuring device 10 to be fixed to the wrist by adhering the second Velcro fastener 320 to the first Velcro fastener 310.

The main body 400 is installed on either the first strap 200 or the second strap 300. The main body 400 performs a function of controlling the operation of the blood pressure measuring device 10 and calculating the subject’s blood pressure, and may include a pump unit 410, a measurement unit 420, a control unit 430, a display unit 440, a power supply unit 460, and a communication unit 470.

The pump unit 410 supplies air to the cuff 100 or suctions air from the cuff 100 to allow air to be discharged.

The measurement unit 420 measures the pressure of the cuff 100, which serves as the basis for calculating blood pressure, and may be implemented using a conventional pressure sensor or the like.

The control unit 430 controls the operation of components constituting the blood pressure measuring device 10. For example, the control unit 430 may control the operation of the cuff 100 so that it pressurizes the subject’s wrist with an appropriate pressure, and may control the display unit 440 to output specific information. In addition, the control unit 430 may calculate the user’s blood pressure based on the pressure measured by the measurement unit 420 and may store the subject’s blood pressure data.

The display unit 440 displays the calculated blood pressure. For example, the display unit 440 may numerically display information regarding the subject’s blood pressure calculated by the control unit 430, but is not limited thereto.

The power supply unit 460 supplies power for operating the blood pressure measuring device 10 and may be implemented using a conventional power source such as a battery.

The communication unit 470 may be implemented using a conventional communication module capable of transmitting and receiving data with an external device, and may employ communication technologies such as Radio Frequency Identification (RFID), Ultra Wide Band (UWB), Bluetooth, or a wireless sensor network suitable for data transmission and reception. Through the communication unit 470, the blood pressure measuring device 10 may transmit the subject’s blood pressure data or an emergency rescue signal to an external device.

An embodiment of the present invention may also be implemented in the form of a computer-readable recording medium containing computer-executable instructions, such as program modules executed by a computer. The computer-readable medium may be any available medium accessible by a computer and may include both volatile and non-volatile media, as well as removable and non-removable media. In addition, the computer-readable medium may include a computer storage medium that stores computer-readable instructions, data structures, program modules, or other data in any method or technology for storage of information, including both volatile and non-volatile, removable and non-removable media.

Although the method and system of the present invention have been described with reference to specific embodiments, some or all of their components or operations may be implemented using a computer system having a general-purpose hardware architecture.

The foregoing description of the present invention is provided for illustrative purposes, and it will be understood by those skilled in the art that various modifications can be made without departing from the technical spirit or essential characteristics of the invention. Therefore, the embodiments described above should be understood as illustrative, not restrictive, in all aspects. For example, each component described as being implemented in a single form may be distributed, and conversely, components described as being distributed may be implemented in an integrated form.

The scope of the present invention is defined by the following claims rather than by the foregoing detailed description, and all modifications or variations derived from the meanings, scope, and equivalents of the claims are to be interpreted as being included within the scope of the present invention.

What is claimed is:

1. A cuff for a blood pressure measuring device, comprising:
an air supply unit including an air inlet through which air is introduced from the outside and an air outlet through which air inside is discharged;
a pressurizing unit connected to the air supply unit and configured to expand when air is introduced from the air supply unit, thereby pressurizing a radial artery located at a wrist of a subject; and
a folding portion disposed between the air supply unit and the pressurizing unit and including a first air passage that connects the air supply unit and the pressurizing unit to communicate with each other;
wherein the pressurizing unit comprises:
a main air bladder disposed to contact the wrist of the subject and connected to the air supply unit;
at least one sub air bladder laminated on one side of the main air bladder; and
a communication passage connecting the main air bladder and the sub air bladder to communicate with each other,
wherein a cross-sectional area of the main air bladder is formed larger than that of the sub air bladder.

2. The cuff for a blood pressure measuring device according to claim 1, further comprising a stent located inside the first air passage of the folding portion, the stent being formed in a coil spring shape.

3. The cuff for a blood pressure measuring device according to claim 1,
wherein the cuff further comprises:
a first branch passage connecting the air supply unit and the first air passage, the first branch passage branching into two flow paths from one end of the first air passage to connect to the air supply unit; and
a second branch passage connecting the first air passage and the main air bladder, the second branch passage branching into two flow paths from the other end of the first air passage to connect to the main air bladder.

4. The cuff for a blood pressure measuring device according to claim 1, further comprising at least one auxiliary air bladder communicating with the air supply unit and located in a direction opposite to the first air passage.

5. The cuff for a blood pressure measuring device according to claim 1,
wherein the folding portion has an extra length such that it is bent in an S-shape when coupled to a strap for wrist wearing.

6. A blood pressure measuring device, comprising:
a first strap configured to wrap around a portion of a subject’s wrist adjacent to an ulnar artery;
a second strap connected to the first strap to wrap around another portion of the wrist adjacent to a radial artery and including the cuff according to claim 1; and
a main body installed on the first strap or the second strap,
wherein the main body comprises:
a pump unit configured to supply air to the cuff or to suction air from the cuff to discharge it;
a measurement unit configured to measure a pressure of the cuff; and
a control unit configured to calculate a user’s blood pressure based on the pressure measured by the measurement unit.

7. The blood pressure measuring device according to claim 6,
wherein the main body further comprises a display unit configured to display the calculated blood pressure.

8. The blood pressure measuring device according to claim 6,
wherein the main body further comprises a communication unit configured to transmit and receive data to and from an external device.
